(19) United States
(12) Patent Application Publication
(10) Pub. No.: US 2024/0058372 A1
(43) Pub. Date: Feb. 22, 2024

(54) ORAL HYALURONIC ACID COMPOSITION

(71) Applicants: Mark Ganaban, Kansas City, MO (US); Darren Landis, Spring Hill, KS (US)

(72) Inventors: Mark Ganaban, Kansas City, MO (US); Darren Landis, Spring Hill, KS (US)

(21) Appl. No.: 18/235,153

(22) Filed: Aug. 17, 2023

Related U.S. Application Data
(60) Provisional application No. 63/398,545, filed on Aug. 17, 2022.

(51) Int. Cl. A61K 31/728; A61K 47/20; A61K 47/12; A61K 47/10; A61K 9/00; A61K 45/06

(52) U.S. Cl. CPC A61K 31/728; A61K 47/20; A61K 47/12; A61K 47/10; A61K 9/0053; A61K 45/06

(57) ABSTRACT

A method for relieving joint pain or other discomforts associated with osteoarthritis in a warm-blooded vertebrate includes delivering to the vertebrate by oral ingestion a nutritional supplement consisting essentially of an effective amount of hyaluronic acid (HA), or a salt of digest thereof, a food acceptable carrier and an additive to maintain a pH of the nutritional supplement above 7.0, wherein an effective amount of HA or salt or digest thereof is from about 0.1 μg per kg to about 400 μg per kg of body weight.

### CROSS-REFERENCE TO RELATED APPLICATIONS

This application claims priority under 35 USC § 119(e) from U.S. Provisional Patent Application No. 63/398,545, filed on Aug. 17, 2022 (“the provisional application”); the content of the provisional patent application is incorporated herein by reference.

### BACKGROUND OF THE INVENTION

Hyaluronic acid (HA), also known as hyaluronan or hyaluronate, is a carbohydrate—more specifically a mucopolysaccharide—occurring naturally throughout the human body. It can be several thousand sugars (carbohydrates) long. When not bound to other molecules, hyaluronic acid binds to water, giving it a stiff viscous quality similar to Jell-O. This viscous gel is one of the most heavily researched substances in medicine with thousands of trials. Its function in the body is, among other things, to bind water and to lubricate movable parts of the body, such as joints. The consistency and tissue-friendliness of viscous HA (with attached water) also allows it to be beneficial in skin-care products, for example, HA can be an excellent moisturizer.

### Hyaluronic Acid in Synovial Fluid

A joint or articulation (or articular surface) is an interface and/or a connection made between bones, ossicles, or other hard structures in the body, which interface and/or connection links the parts of the skeletal system (most mammalian skeletal systems, particularly human) into a functional whole.

Joints (like human elbows and knees) are surrounded by a membrane called the synovial membrane. Synovial membranes form capsule-like enclosures or semi-enclosures around the ends of the two articulating bones. Synovial membrane secrete a liquid called synovial fluid. Synovial fluid is viscous, having a fluid consistency or viscosity quite similar to that of motor oil. Synovial fluid performs many functions, but none more important than providing the elastic shock absorbing properties of mammalian joints. In the joints, synovial fluid provides additional functions, such as carrying nutrients to the cartilage, removing waste from the joint capsule, etc., as known to persons of ordinary skill in the art.

### Hyaluronic Acid in Gum Tissue

Mammalian gums (gingivoe) are composed of dense fibrous connective tissue (ligaments) which secure the teeth to the alveolar bone (jawbone). Connective tissue is composed of a fibrous tissue surrounded by Hyaluronic Acid (extra-cellular matrix). Hyaluronic acid helps support tensile strength of the ligaments that secure the teeth in place in the jaw. The hyaluronic acid does this by providing hydration and nourishment to the gum tissue. The result is a healthy set of gums. If mammalian gums are deprived of Hyaluronic Acid, the physical state of the gums degrades shortly resulting in a very unhealthy state.

In relatively recent times, many hyaluronic acid (HA) based products have developed and have become available to consumers.

SYNTHOVIAL SEVEN® joint support product, by Hyalogic LLC, Riverside, Missouri, for example, supports joint health by providing high molecular weight HA to the user's body as a dietary supplement (e.g., Hyalogic's HA Chewable Lozenges). Developing Hyalogic's HA products, including the SYNTHOVIAL SEVEN® joint support product, took many rounds of research and testing. But the result of the research and testing realized a liquid hyaluronic acid product delivered real joint health benefits.

In 2000, inventors Darren Landis and Mark Ganaban founded Hyalogicz®, Inc. Hyalogic is a producer of an oral liquid Hyaluronic Acid (HA) product for supplementation. Hyalogic®, Inc. has been providing the liquid HA products such as SYNTHOVIAL SEVEN® joint support product since that time.

It is found, however, that acidic drinks (such as pH hyaluronic acid products exhibiting a pH of less than 7 may be harmful to the user's teeth. Enamel, the outer covering of teeth, while being the hardest substance in the body, can be damaged by acid or an acidic environment. Liquids with lower pH (i.e., an acidic pH) display the lower pH because the liquids contain more acid. The lower the pH goes, the more acidic the liquid. Hence, the lower the pH the higher the potential to tooth enamel and, therefore, greater damage to the teeth. And as is known to the skilled person, sugar-sweetened beverages like soda are some of the most acidic liquids.

### SUMMARY OF THE INVENTION

The following detailed description of embodiments of the invention will be made in reference to the accompanying drawings. In describing the invention, explanation about related functions or constructions known in the art are omitted for the sake of clearness in understanding the concept of the invention to avoid obscuring the invention with unnecessary detail.

In an embodiment, the invention provides a hyaluronic acid product for oral administration that supports not only the health of mammalian hair, skin, eyes and bones, but also supports joint health while protecting teeth and gums from deleterious effects of composition constituents of other known HA products that may be acidic, where a consumption of such acidic HA products might damage tooth enamel.

The inventive HA product and method of oral administration of same described herein can be manufactured with HA that is commercially available. Typically, the HA used in products manufactured according to the invention is derived from bacterial organisms. The HA preferably is polydiverse, falling within a molecular weight range of between around 50,000 Daltons and 8,000,000 Daltons, depending on the source and method of isolation. The pH of the inventive HA products is at least 7.0, so that the actual product HA-containing liquid contacting the consumer's teeth and gums is not acidic, so cannot damage either.

In an embodiment, the invention provides a method for relieving joint pain or other discomforts associated with osteoarthritis in a warm-blooded vertebrate. The method includes delivering to said vertebrate, by oral ingestion, a nutritional supplement consisting essentially of an effective amount of hyaluronic acid (HA), or a salt or digest thereof, a food acceptable carrier and a pH-affecting additive to maintain a pH of the nutritional supplement above 7.0. An effective amount of the HA, or a salt or digest thereof, is included in the nutritional supplement in a range of about 0.1 μg per kg of body weight to about 400 μg per kg of body weight.

The nutritional supplement may also include an additive to maintain an overall health of gum and teeth of the warm-blooded vertebrate to which the nutritional supplement is delivered; the additive may be xylitol. The pH-affecting additive that maintains the pH of the nutritional supplement above 7.0 may be sodium hydroxide, sodium citrate, lemon juice, baking soda, pH drops, ionized water, etc., without limitation, as known to those of ordinary skill in the art. In the method, the effective amount of hyaluronic acid is 2 mg to 250 mg per dose preferably 100 mg/dose. In an embodiment, the invention provides a method supporting and maintaining health of hair, skin, eyes, bones and joints in a warm-blooded vertebrate, the supporting and maintaining joint health including hydrating the hair, skin, eyes and joints. The method includes delivering to the warm-blooded vertebrate, by oral ingestion, a nutritional supplement comprising an effective amount of hyaluronic acid (HA), or a salt of digest thereof, and a food acceptable carrier, A pH of the nutritional supplement is maintained above 7.0 in the method. An effective amount of the HA or salt or digest thereof is from about 0.1 μg per kg to about 400 μg per kg of body weight, preferably 10 μg per kg of body weight. The nutritional supplement preferably comprises an additive to maintain an overall health of gum and teeth of the warm-blooded vertebrate, The further additive may be Xylitol. The nutritional supplement may be maintained at a pH above 7.0 by addition of an alkaline substance.

In an embodiment, the invention provides a method for supporting cognitive health in humans. The method includes delivering to said human, by oral ingestion, a nutritional supplement consisting essentially of an effective amount of hyaluronic acid (HA), or a salt or digest thereof, a food acceptable carrier and a pH-affecting additive to maintain a pH of the nutritional supplement above 7.0. An effective amount of the HA, or a salt or digest thereof, preferably is included in the nutritional supplement in a range of about 0.1 μg per kg of body weight to about 400 μg per kg of body weight. The delivered nutritional supplement additionally supports emotional or mood stability in the humans.

It will be appreciated by those skilled in the art that the foregoing brief description and the following detailed description of the invention are exemplary and explanatory of the invention. The descriptions combined, however, are not intended to be restrictive or limiting of the advantages which can be achieved by the invention.

### DETAILED DESCRIPTION OF THE INVENTION

The following is a detailed description of example embodiments of the invention, which are presented in such detail as to clearly communicate the invention and are designed to make such embodiments obvious to a person of ordinary skill in the art. However, the amount of detail offered is not intended to limit the contemplated variations of the embodiments of the disclosed invention. On the contrary, the inventor intends to cover all modifications, equivalents, and alternatives falling within the spirit and scope of the disclosed invention, as defined by the appended claims.

The inventive hyaluronic acid (HA) product embodies a liquid composition comprising bacterially synthesized hyaluronan polymers with different molecular weights in a range of 50 k to 8M Daltons. The liquid composition further comprises a pharmaceutically acceptable carrier, such as water, and an additive for reducing the chance that consuming the inventive HA will result in enamel degradation and consequential cavities, The pH of the inventive liquid composition is at least 7.0, so that it is not acidic, for example, 7.5.

Xylitol may be used as the additive. That is, xylitol may be added to the liquid HA composition to protect the gum tissue of consumers who consume the HA product and limit dental cavities of the teeth surrounded by the gums. With additives that minimize inadvertent tooth enamel damage to consumers, such as xylitol, the inventive HA product thereby protects the consumer's teeth and gums while supporting joint health, as explained above. Preferably, the inventive HA products with teeth and gum protection provide HA in a range of about 2 mg to about 250 mg. In one embodiment, the HA is included in an amount of 100 mg.

The liquid HA product is prepared by mixing polydiverse HA in a molecular weight range between around 50,000 Daltons and 8,000,000 Daltons, depending on the source and method of isolation, to a food acceptable carrier, such as water. Xylitol for tooth and gum protection may then be added, depending on the product formula. In some cases, a second additive may be added to the compositions at that stage in order to maintain the pH above 7.0. As explained, at a pH above 7.0, the HA product will not damage the teeth and gums of consumers. Additional elements can be included in the composition in varying amounts, such as slippery elm, cranberry seed extract, fruit pectin, etc., for added tooth protection, for vitamin administrations, or even for medical treatments, depending on the additive.

The inventive HA composition with teeth and gum protection provides relief from joint pain and musculoskeletal discomfort in vertebrates suffering from arthritic conditions such as osteoarthritis in warm-blooded vertebrates and protects their gums from what might otherwise be the deleterious effects of acidic HA-based supplements.

The inventive HA compositions, after consumption, are absorbed during the digestive process and are thereby effectively available to relieve joint discomfort with a short period of time.

### Example 1

The following is an exemplary embodiment presented for illustrative purposes only, which is not meant to be limiting.

The inventive HA composition may be manufactured in scale according to the following process and formula, which process describes quantities required for a forming or manufacturing a serving, for example, a 10 ml serving will deliver about 100 mg of HA and 100 mg of MSM (Methylsulfonylmethane), in one inventive formula [[what is MSM?]. The ingredients listed below are combined in distilled water in a beaker (e.g., a Griffin beaker). The amounts of sodium citrate, potassium sorbate and vegetable glycerine may be varied to assure a pH of at least 7.0, as they are inactive ingredients:
a. 100 mg HA;
b. 100 mg of MSM (Methylsulfonoymethane);
c. Sodium Citrate;
d. Potassium Sorbate; and
e. Vegetable Glycerine.

In an embodiment, the invention provides a method for relieving joint or musculoskeletal pain or discomfort by delivering to the consumer by oral ingestion a composition comprising an effective amount of hyaluronic acid, or a salt or digest thereof, a nutritionally acceptable carrier, an additive to promote teeth and gum health such as xylitol and an additive for maintaining the liquid composition at a pH as least as high as 7.0.

An effective amount as used herein refers to an amount of hyaluronic acid which, upon oral administration, provides relief of joint pain of discomfort without damaging tooth enamel. The effective amount of hyaluronic acid, or a salt of digest thereof, is from about 0.1 microgram/kilogram to about 400 microgram/kilogram body weight per dose

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. A method for relieving joint pain or other discomforts associated with osteoarthritis in a warm-blooded vertebrate comprising the step of:
delivering to said vertebrate, by oral ingestion, a nutritional supplement consisting essentially of an effective amount of hyaluronic acid (HA), or a salt or digest thereof, a food acceptable carrier and a pH-affecting additive to maintain a pH of the nutritional supplement above 7.0,
wherein an effective amount of the HA, or a salt or digest thereof, is included in the nutritional supplement in a range of about 0.1 μg per kg of body weight to about 400 μg per kg of body weight.

2. The method of claim 1, wherein the nutritional supplement further consists of a further additive to maintain an overall health of gum and teeth of the warm-blooded vertebrate to which the nutritional supplement is delivered.

3. The method of claim 2, wherein the further additive is any of the group of additive including xylitol, slippery elm, cranberry seed extract, fruit pectin.

4. The method of claim 1, wherein the pH-affecting additive that maintains the pH of the nutritional supplement above 7.0 is sodium hydroxide.

5. The method of claim 1, wherein the pH-affecting additive that maintains the pH of the nutritional supplement above 7.0 is sodium citrate.

6. The method of claim 1, wherein the effective amount of hyaluronic acid is 2 mg to 250 mg per dose.

7. A method for supporting and maintaining health of hair, skin, eyes, bones, brain, heart, connective tissue and joints in a warm-blooded vertebrate, the supporting and maintaining joint health including hydrating the hair, skin, eyes and joints, the method comprising at least one step of:
delivering to the warm-blooded vertebrate, by oral ingestion, a nutritional supplement comprising an effective amount of hyaluronic acid (HA), or a salt of digest thereof, and a food acceptable carrier;
wherein a pH of the nutritional supplement is maintained above 7.0, and
wherein an effective amount of the HA or salt or digest thereof is from about 0.1 μg per kg to about 400 μg per kg of body weight.

8. The method of claim 7, wherein the nutritional supplement comprises an additive to maintain an overall health of gum and teeth of the warm-blooded vertebrate

9. The method of claim 8, wherein the further additive is Xylitol.

10. The method of claim 7, wherein the nutritional supplement is maintained at a pH above 7.0 by addition of an alkaline substance.

11. A hyaluronic acid-based (HA-based) nutritional supplement composition, comprising:
purified water;
100 mg HA;
100 of Methylsulfonoymethane (MSM);
sodium citrate;
potassium sorbate;
vegetable glycerine;
wherein, the HA-based nutritional supplement composition displays a pH greater than 7.0.

12. The hyaluronic acid-based (HA-Based) nutritional supplement composition of claim 11, wherein one or more of the sodium citrate, potassium sorbate and vegetable glycerine effects the pH of greater than 7.0.
